(19) United States
(12) Patent Application Publication
(10) Pub. No.: US 2016/0032276 A1
(43) Pub. Date: Feb. 4, 2016

(54) SYSTEMS AND METHODS FOR ISOLATING NUCLEIC ACIDS

(71) Applicant: IBIS BIOSCIENCES, INC., Carlsbad, CA (US)

(72) Inventor: Mark W. Eshoo, Carlsbad, CA (US)

(73) Assignee: IBIS BIOSCIENCES, INC., Carlsbad, CA (US)

(21) Appl. No.: 14/775,052

(22) PCT Filed: Mar. 12, 2014
(86) PCT No.: PCT/US14/24504

§ 371 Date: Sep. 11, 2015

Related U.S. Application Data
(60) Provisional application No. 61/782,630, filed on Mar. 14, 2013.

(51) Int. Cl. C12N 15/10; C12Q 1/68

(52) U.S. Cl. CPC C12N 15/1006; C12Q 1/6806

(57) ABSTRACT

The present disclosure relates to systems and methods for nucleic acid isolation. In particular, the present disclosure provides systems and methods for purifying nucleic acids for downstream applications.

This application claims priority to Provisional Patent Application Ser. No. 61/782,630, filed Mar. 14, 2013; which is herein incorporated by reference in its entirety.

### FIELD OF INVENTION

The present disclosure relates to systems and methods for nucleic acid isolation. In particular, the present disclosure provides systems and methods for purifying nucleic acids for downstream applications.

### BACKGROUND

Nucleic acids found in cells can be deoxyribonucleic acid or ribonucleic acid and can be genomic DNA, extrachromosomal DNA (e.g. plasmids and episomes), mitochondrial DNA, messenger RNA and transfer RNA. Nucleic acids can also be foreign to the host and contaminate a cell as an infectious agent, e.g. bacteria, viruses, fungi or single celled organisms and infecting multicellular organisms (parasites). Recently, detection and analysis of the presence of nucleic acids has become important for the identification of single nucleotide polymorphisms (SNPs), chromosomal rearrangements and the insertion of foreign genes. These include infectious viruses, e.g. HIV and other retroviruses, jumping genes, e.g. transposons, and the identification of nucleic acids from recombinantly engineered organisms containing foreign genes.

The analysis of nucleic acids has a wide array of uses. For example, the presence of a foreign agent can be used as a medical diagnostic tool. The identification of the genetic makeup of cancerous tissues can also be used as a medical diagnostic tool, confirming that a tissue is cancerous, and determining the aggressive nature of the cancerous tissue. Chromosomal rearrangements, SNPs and abnormal variations in gene expression can be used as a medical diagnostic for particular disease states. Further, genetic information can be used to ascertain the effectiveness of particular pharmaceutical drugs, known as the field of pharmacogenomics. Genetic variations between humans and between domestic animals can also be ascertained by DNA analysis. This is used in fields including forensics, paternity testing and animal husbandry.

Methods of extracting nucleic acids from cells are well known to those skilled in the art. A cell wall can be weakened by a variety of methods, permitting the nucleic acids to extrude from the cell and permitting its further purification and analysis. The specific method of nucleic acid extraction is dependent on the type of nucleic acid to be isolated, the type of cell, and the specific application used to analyze the nucleic acid. Many methods of isolating DNA are known to those skilled in the art, see for example the general reference Sambrook and Russell, 2001, “Molecular Cloning: A Laboratory Manual”. For example, the prior art contains examples of chemically-impregnated and dehydrated solid-substrates for the extraction and isolation of DNA from bodily fluids that employ lytic salts and detergents and which contain additional reagents for long-term storage of DNA samples e.g. U.S. Pat. No. 5,807,527 detailing FTA paper and U.S. Pat. No. 6,168,922 detailing Isocard Paper. The prior art also contains examples of particle separation methods, e.g. U.S. RE 37,891.

While many nucleic acid purification procedures are well known and have been in existence for years, these procedures can be time consuming and may employ reagents that present dangers to those performing the purification. For example, it has long been known that DNA and RNA readily can be obtained in a purified form from a test sample using organic extraction procedures, but such procedures can require several extractions and therefore can be time consuming. Additionally, the use of organic solvents is undesirable and dangerous if proper precautions are not followed.

Accordingly, there is a need for a safe, effective and convenient method for isolating and purifying nucleic acids.

### SUMMARY

The present disclosure relates to systems and methods for nucleic acid isolation. In particular, the present disclosure provides systems and methods for purifying nucleic acids for downstream applications.

Embodiments of the present disclosure provide systems and methods that reduce ethanol carry over from nucleic acid purification to downstream applications. The wash buffers with reduced ethanol described herein improve downstream applications without reducing yield of nucleic acids.

For example, in some embodiments, the present invention provides a method of isolating nucleic acids, comprising: a) contacting a sample comprising said nucleic acids with a solid support such that the nucleic acids bind to the solid support; b) washing the solid support with a wash buffer comprising a surfactant and 35% or less ethanol such that contaminants are removed from the solid support and nucleic acids are retained on the solid support; and c) eluting nucleic acids from the solid support. In some embodiments, the method further comprises performing a downstream analysis assay on the nucleic acid (e.g., sequencing, amplification, detection, etc.).

The present invention is not limited to particular concentrations of ethanol in the wash buffer. For example, in some embodiments, the wash buffer comprises 30%, 20%, 15%, 10%, 5% or less ethanol. In some embodiments, the wash buffer comprises 35%, 30%, 20%, 10%, 5% or zero ethanol.

In some embodiments, wash buffers comprise between 10% and 50% surfactant (e.g., 10%, 15%, 20%, 25%, 30%, 35%, or 40% surfactant). In some embodiments, the surfactant is polysorbate 20.

In some embodiments, the wash buffer comprises a salt (e.g., MgCl). In some embodiments, the salt is present at a concentration of between 5 and 15 mM (e.g., 10 mM).

In some embodiments, buffer has a pH of between 3.5 and 5.0 (e.g., 5.0). In some embodiments, buffer comprises components selected from, for example, 40% Tween 20, 35% Ethanol, 25% water; 20% Tween 20, 20% Ethanol, 60% water; 10% Tween 20, 35% Ethanol, 55% water; 20% Tween 20, 0% ethanol, 80% water; 35% Tween 20, 30% ethanol, 45% water; 20% Tween 20, 20% ethanol, 60% water+10 mM MgCl; 20% Tween 20, 10% ethanol, 70% water+10 mM MgCl; 30% Tween 20, 5% ethanol, 65% water+10 mM MgCl; or 30% Tween 20, 0% ethanol, 70% water+10 mM MgCl.

The present invention is not limited to a particular sample or nucleic acid. The methods described herein are suitable for isolation of DNA or RNA from a variety of samples and sources. For example, in some embodiments, the sample is a cell lysate (e.g., mammalian, fungal, viral or bacterial cell), blood, serum, or urine. In some embodiments, the nucleic acid is mammalian, viral, fungal, bacteria, or synthetic nucleic acid.

The present invention also provides a kit, comprising: a) a wash buffer comprising a surfactant and 35% or less ethanol; and b) a solid support. The present invention is not limited to a particular solid support. Examples include, but are not limited to, a resin, a column, a particle, or a bead.

Additional embodiments of the present invention provide a composition, comprising a nucleic acid non-covalently bound to a solid support; and a wash buffer comprising a surfactant and 35% or less ethanol.

The present invention further provides a method of isolating nucleic acids, comprising: a) contacting a sample comprising nucleic acids with a solid support such that the nucleic acids bind to the solid support; b) washing the solid support with a wash buffer comprising 20% polysorbate 20, 10% ethanol, and 10 mM MgCl, wherein the buffer has a pH of 5.0, such that contaminants are removed from the solid support and nucleic acids are retained on the solid support; and c) eluting the nucleic acids from the solid support.

The present invention additionally provides a method of isolating RNA, comprising: a) contacting a sample comprising RNA with a solid support such that the RNA binds to the solid support; b) washing the solid support with a wash buffer comprising 30% polysorbate 20, 5% ethanol, and 10 mM MgCl, wherein the buffer has a pH of 5.0, such that contaminants are removed from the solid support and the RNA is retained on the solid support; and c) eluting the RNA from the solid support.

The present invention further provides a method of isolating RNA, comprising: a) contacting a sample comprising RNA with a solid support such that the RNA binds to the solid support; b) washing the solid support with a wash buffer comprising 30% polysorbate 20 and 10 mM MgCl, wherein said buffer has a pH of 5.0, and wherein the buffer lacks ethanol, such that contaminants are removed from the solid support and said RNA is retained on the solid support; and c) eluting the RNA from the solid support.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 shows Kp DNA and MS2 RNA recovery using wash buffers of embodiments of the present disclosure.

FIG. 2 shows Kp DNA and MS2 RNA recovery using wash buffers of embodiments of the present disclosure.

FIG. 3 shows Kp DNA and MS2 RNA recovery using wash buffers of embodiments of the present disclosure.

FIG. 4 shows Kp DNA and MS2 RNA recovery using wash buffers of embodiments of the present disclosure.

FIG. 5 shows Kp DNA and MS2 RNA recovery using wash buffers of embodiments of the present disclosure.

FIG. 6 shows agarose gel quantitation of genomic DNA recovery using wash buffers of embodiments of the present disclosure.

FIG. 7 shows Kp DNA and MS2 RNA obtained from PCR amplification after purification using wash buffers of embodiments of the present disclosure.

FIG. 8 shows Kp DNA and MS2 RNA obtained from PCR amplification after purification using wash buffers of embodiments of the present disclosure.

FIG. 9 shows Kp DNA and MS2 RNA obtained from PCR amplification after purification using wash buffers of embodiments of the present disclosure.

FIG. 10 shows Kp DNA and MS2 RNA recovery using wash buffers of embodiments of the present disclosure.

FIG. 11 shows Kp DNA and MS2 RNA recovery from virus and bacteria using wash buffers of embodiments of the present disclosure.

FIG. 12 shows Kp DNA and MS2 RNA recovery using wash buffers of embodiments of the present disclosure.

FIG. 13 shows Kp DNA and MS2 RNA recovery using wash buffers of embodiments of the present disclosure.

FIG. 14 shows Kp DNA and MS2 RNA recovery from virus and bacteria using wash buffers of embodiments of the present disclosure.

FIG. 15 shows Kp DNA and MS2 RNA obtained from PCR amplification after purification using wash buffers of embodiments of the present disclosure.

FIG. 16 shows Kp DNA and MS2 RNA obtained from PCR amplification after purification using wash buffers of embodiments of the present disclosure.

FIG. 17 shows Kp DNA and MS2 RNA recovery using wash buffers of embodiments of the present disclosure.

FIG. 18 shows Kp DNA and MS2 RNA recovery using wash buffers of embodiments of the present disclosure.

### DETAILED DESCRIPTION

The present disclosure relates to systems and methods for nucleic acid isolation. In particular, the present disclosure provides systems and methods for purifying nucleic acids for downstream applications.

### Definitions

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “amplicon” refers to a nucleic acid generated via amplification reaction. The amplicon is typically double stranded DNA; however, it may be RNA and/or DNA:RNA. The amplicon comprises DNA complementary to a sample nucleic acid. In some embodiments, primer pairs are configured to generate amplicons from a sample nucleic acid. As such, the base composition of any given amplicon may include the primer pair, the complement of the primer pair, and the region of a sample nucleic acid that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used for subsequent analysis (e.g. base composition determination). In some embodiments, the amplicon further comprises a length that is compatible subsequent analysis.

The term “amplifying” or “amplification” in the context of nucleic acids refers to the production of multiple copies of a polynucleotide, or a portion of the polynucleotide, typically starting from a small amount of the polynucleotide (e.g., as few as a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “solid support” refers to a substrate or other solid material that does not dissolve in aqueous solutions utilized in nucleic acid purification or isolation. For example, in some embodiments, solid supports are substrates utilized in nucleic acid purification and isolation. Examples include, but are not limited to, beads, particles, resins, chromatography columns, and the like. In some embodiments, solid supports are coated or functionalized with material that enhances nucleic acid binding.

As used herein, the terms “subject” and “patient” refer to any animal, such as a dog, a cat, a bird, livestock, and particularly a mammal, and preferably a human.

As used herein, the term “sample” is used in its broadest sense. In one sense, it is meant to include a representative portion or culture obtained from any source, including biological and environmental sources. Biological samples may be obtained from animals (including humans) and encompass fluids, solids, tissues, and gases. Biological samples include blood products, such as plasma, serum, and the like. Environmental samples include environmental material such as surface matter, soil, mud, sludge, biofilms, water, and industrial samples. Such examples are not however to be construed as limiting the sample types applicable to the present invention.

### Embodiments of the Technology

Although the disclosure herein refers to certain illustrated embodiments, it is to be understood that these embodiments are presented by way of example and not by way of limitation.

Ethanol carryover from nucleic acid isolation can negatively affect downstream processes. Many current nucleic acid isolation techniques require a wash with a high concentration of ethanol in order to wash away contaminates while keeping the nucleic acids bound to a capture substrate. Embodiments of the present disclosure address this problem by providing wash systems and methods that use a reduced amount of ethanol. This lowers the ethanol carry-over to downstream applications.

Reducing the amount of ethanol in the wash solution used to wash away contaminates from nucleic acid purification reactions reduces the amount of ethanol that is carried to downstream applications. Since many of these applications perform poorly in the presence of ethanol a reduction in ethanol carry-over increases performance in the downstream applications.

### I. Buffers and Purification Systems and Methods

Many commercial nucleic acid purification kits and systems function by binding nucleic acids to a solid support (e.g., column, bead, particle and the like). Contaminants are removed by washing with a wash buffer. Purified nucleic acids are then eluted from the support. Wash buffers frequently contain ethanol, which can impede downstream applications (e.g., nucleic acid analysis).

Ethanol is included in wash buffer because it is effective at promoting binding of nucleic acids to solid supports and removal of salts during wash steps. This is due to ethanol's polar nature and hydrogen bonding with waters, which promotes DNA binds and dissolves low MW salts and detergents.

Accordingly, embodiments of the present disclosure provide systems and methods that allow for purification and isolation of nucleic acids with reduced ethanol wash buffers and consequently, decreased ethanol carry over to downstream applications.

In some embodiments, the present invention provides a buffer (e.g., wash buffer) for eluting a nucleic acid (e.g., DNA or RNA) from a solid support. In some embodiments, the buffer comprises less than 70% ethanol by volume (e.g., approximately or about 65%, 60%, 55%, 50%, 45%, 40%, 35%, 30%, 25%, 20%, 15%, 10% or less ethanol; + or − 1%, 2%, 3%, 4%, or 5%). For example, in some embodiments, the buffer comprises between approximately 5% and 50%, approximately 5% and 40%, approximately 5% and 30%, approximately 5% and 20%, approximately 5% and 10%, approximately 10% and 30%, approximately 10% and 25%, approximately 10% and 20%, approximately 10% and 15%, approximately 15% and 45%, approximately 20% and 40%, approximately 20% and 30%, or approximately 25% and 35% ethanol; + or − 1%, 2%, 3%, 4%, or 5%. In some embodiments, the buffer comprises approximately 35% ethanol. In some embodiments, the buffer comprises approximately 20% ethanol.

In some embodiments, the buffer comprises a surfactant (e.g., non-ionic surfactant). In some embodiments, the surfactant is Polysorbate 20 (trade name Tween 20). In some embodiments, the buffer contains between approximately 10% and 50% surfactant by volume (e.g., approximately 10%, 15%, 20%, 25%, 30%, 35%, 40%, 45%, or 50%; + or − 1%, 2%, 3%, 4%, or 5%). In some embodiments, the buffer contains approximately 20% or 30%; + or − 1%, 2%, 3%, 4%, or 5% Tween 20 surfactant.

In some embodiments, the buffer contains salts. For example, in some embodiments, the buffer contains MgCl at a mM concentration (e.g., 5-15 mM (e.g., 10 mM)).

In some embodiments, the pH of the buffer is 5 or lower (e.g., 5.0, 4.5, 4.0, 3.5, 3.0, or fractions thereof). In some embodiments, the pH of the buffer is 5.0.

Buffers may contain additional components useful, necessary, or sufficient for their intended function. For example, in some embodiments, buffers contain additional salts, detergents, and buffering agents. Suitable buffer agents are commercially available from a number of sources (e.g., from Sigma-Aldrich, St. Louis, Mo.). Exemplary buffers include, but are not limited to, TAPS (3-{[tris(hydroxymethyl)methyl]amino}propanesulfonic acid), Bicine (N,N-bis(2-hydroxyethyl)glycine), Tris (tris(hydroxymethyl)methylamine), Tricine (N-tris(hydroxymethyl)methylglycine), TAPSO (3-[N-Tris(hydroxymethyl)methylamino]-2-hydroxypropanesulfonic Acid), HEPES (4-2-hydroxyethyl-1-piperazineethanesulfonic acid), TES (2- {[tris(hydroxymethyl)methyl]amino}ethanesulfonic acid), MOPS (3-(N-morpholino)propanesulfonic acid), PIPES (piperazine-N,N′-bis(2-ethanesulfonic acid), Cacodylate (dimethylarsinic acid), SSC (saline sodium citrate), MES (2-(N-morpholino)ethanesulfonic acid) and Succinic acid. In some embodiments, wash buffers comprising the following are utilized:
40% Tween 20, 35% Ethanol, 25% water
20% Tween 20, 20% Ethanol, 60% water
10% Tween 20, 35% Ethanol, 55% water
20% Tween 20, 0% ethanol, 80% water
35% Tween 20, 30% ethanol, 45% water pH 5.0
20% Tween 20, 20% ethanol, 60% water+10 mM MgCl
20% Tween 20, 10% ethanol, 70% water+10 mM MgCl; pH 5
30% Tween 20, 5% ethanol, 65% water+10 mM MgCl; pH5
30% Tween 20, 0% ethanol, 70% water+10 mM MgCl; pH5

The present disclosure is not limited to a particular source of nucleic acids for isolation. The systems and methods described herein are suitable for use in the isolating and purification of both DNA and RNA. DNA and RNA can be isolated simultaneously or separately, depending on the application. In some embodiments, nucleic acids are mammalian (e.g., genomic). In other embodiments, nucleic acids from foreign pathogens (e.g., viruses, bacteria, fungi, etc.) are isolated. In some embodiments, nucleic acids are synthetic.

In some embodiments, the nucleic acid isolation comprises the following steps: a) breaking cell walls (e.g., using any suitable method including but not limited to bead beating in the presence of detergent); b) contacting lysate with a solid support that binds the nucleic acid of interest, c) washing the solid support with a wash buffer (e.g., those described herein) to remove contaminants but retain the nucleic acid; and d) eluting the nucleic acid from the solid support.

The present disclosure is not limited to a particular solid support. In some embodiments, commercially available solid supports or isolation systems are utilized. In some embodiments, solid supports are beads, columns, or particles.

In some embodiments, one or more steps are automated (e.g., using automated sample handling or robotics).

### II. Downstream Applications

Following isolation, nucleic acids may be analyzed using any suitable method. In some embodiments, the presence of pathogens is detected. In other embodiments, the presence of nucleic acid variants, polymorphisms, mutations, copy number variations, methylation status, etc. are detected.

Examples of nucleic acid detection methods include, but are not limited to, sequencing, amplification, microarrays, probe binding and the like. Exemplary methods are described below.

### A. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined. In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671956; U.S. patent application Ser. No. 11/781166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

### B. Hybridization

Illustrative non-limiting examples of nucleic acid hybridization techniques include, but are not limited to, microarrays including, but not limited to: DNA microarrays (e.g., cDNA microarrays and oligonucleotide microarrays). A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

### C. Amplification

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

### D. Detection Methods

Non-amplified or amplified nucleic acids can be detected by any conventional means. For example, the nucleic acids can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

In some embodiments, nucleic acids are detected and characterized by the identification of a unique base composition signature (BCS) using mass spectrometry (e.g., Abbott PLEX-ID system, Abbot Ibis Biosciences, Abbott Park, Ill.,) described in U.S. Pat. Nos. 7,108,974, 8,017,743, and 8,017,322; each of which is herein incorporated by reference in its entirety.

In some embodiments, a computer-based analysis program is used to translate the raw data generated by the detection assay (e.g., the presence, absence, or amount of a given nucleic acid) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a nucleic acid) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### E. Systems and Kits

In some embodiments, the present invention provides kits and systems for the lysis, isolation, and analysis of nucleic acids (e.g., DNA and/or RNA). In some embodiments, kits include reagents necessary, sufficient or useful for detection of nucleic acids (e.g., reagents, wash buffers, controls, instructions, etc.). In some embodiments, kits comprise solid supports for binding nucleic acids (e.g., beads, resins, columns, particles, etc.).

In some embodiments, kits comprise one or more containers that comprise reagents, solid supports, buffers (e.g., wash buffers, lysis buffers, elution buffers, etc.), controls, and the like. In some embodiments, each component of the kit is packaged in a separate container. In some embodiments, the containers are packed and/or shipped in the same kit or box for use together. In some embodiments, one or more components of the kit are shipped and/or packaged separately.

In some embodiments, systems include automated sample and reagent handling devices (e.g., robotics).

### EXPERIMENTAL

The following examples are provided in order to demonstrate and further illustrate certain preferred embodiments and aspects of the present invention and are not to be construed as limiting the scope thereof.

### EXAMPLE 1

Wash substitutes for 70% Ethanol
Standard Column Isolation Protocol (SCIP)
This is a benchtop protocol for the isolation of nucleic acids (DNA and RNA).
Reagents and consumables:
Qiagen DNEasy columns
Qiagen collection tube for columns
Poly A (10 mg/mL) (Ibis MG-00325)
Wash 1 (Abbott MG-00371)
100% Ethanol
Primer Dilution Solution (Ibis MG-00051)
1.4× A10 Complete or AVE solution
Prepare Lysis Solution C1: Mix Wash 1 with 100% ethanol in a 1:1 ratio.
Prepare 1× A10 Complete (if used): Dilute the 1.4× A10 Complete to 1× with Primer Dilution Solution.
Wash solution: depends upon condition being tested.
Protocol:
1. Add 400 uL of Lysis Solution C1 to 200 uL of sample and mix (A 2:1 ratio of Lysis Solution C1 to sample).
2. Load lysate onto DNEasy column. Spin 1 min at 6 k * g and transfer column to new collection tube.
3. Add 500 uL of Wash Solution to capture column. Spin 1 min at 6 k * g and transfer column to new collection tube.
4. Repeat step 3.
5. Transfer column to 1.5 mL snapcap tube and add 100 uL of 1× A10 Complete OR AVE solution and incubate at room temperature for 3 min.
6. Spin for 1 min at 6 k * g to elute nucleic acids.
Alternatives to using 70% Ethanol wash were tested. Both PEG and non-ionic detergent TWEEN were tested for their ability to use reduced percentage of ethanol in the wash solution while retaining the nucleic acids on the capture substrate.

| Condition | Composition |
|---|---|
| Normal | 70% ethanol, 30% Primer Dilution Solution |
| T20 | 40% Tween 20, 35% ethanol, 25% Water |
| T40 | 40% Tween 40, 35% ethanol, 25% Water |
| T80 | 40% Tween 80, 35% ethanol, 25% Water |
| PEG | 8% PEG8000 (w/w), 35% ethanol, 57% Water |

Spiked sample was made with K. pneumonia (Kp) DNA and MS2 RNA as follows:

| 1.00E+07 | cp MS2 per extraction |
|---|---|
| 1.00E+09 | cp MS2 RNA/uL stock |
| | 1:100 dilution MS2 |
| 1.00E+07 | cp MS2 RNA/uL after dilution |
| 1.00E+04 | cp Kp per extraction |
| 5 | fg per Kp genome |
| 5.00E+04 | fg Kp DNA per extraction |
| 5.00E+01 | pg Kp DNA per extraction |
| 10000 | Stock Kp concentration pg/uL |
| | 1:100 dilution of Kp stock |
| 100 | pg/uL Kp after dilution |
| | ug/uL PolyA |
| 10 | stock ug per |
| 5 | extraction |

Sample Stock Prep

| | |
|---|---|
| 16 | # of extractions |
| 200 | volume of extraction (uL) |
| 8 | uL PolyA stock |
| 16.00 | uL MS2 RNA dilution |
| 8 | uL Kp DNA dilution |
| 3200 | volume PDB |

Two replicates of each Wash Solution were tested. Nucleic acids were isolated according to the SCIP with each wash solution condition being used. Samples were eluted with AVE solution. The amount of Kp DNA and MS2 RNA was quantitated with qPCR and qRT-PCR respectively. Total copies of Kp genome and MS2 genome recovered using each of the wash solutions was compared.

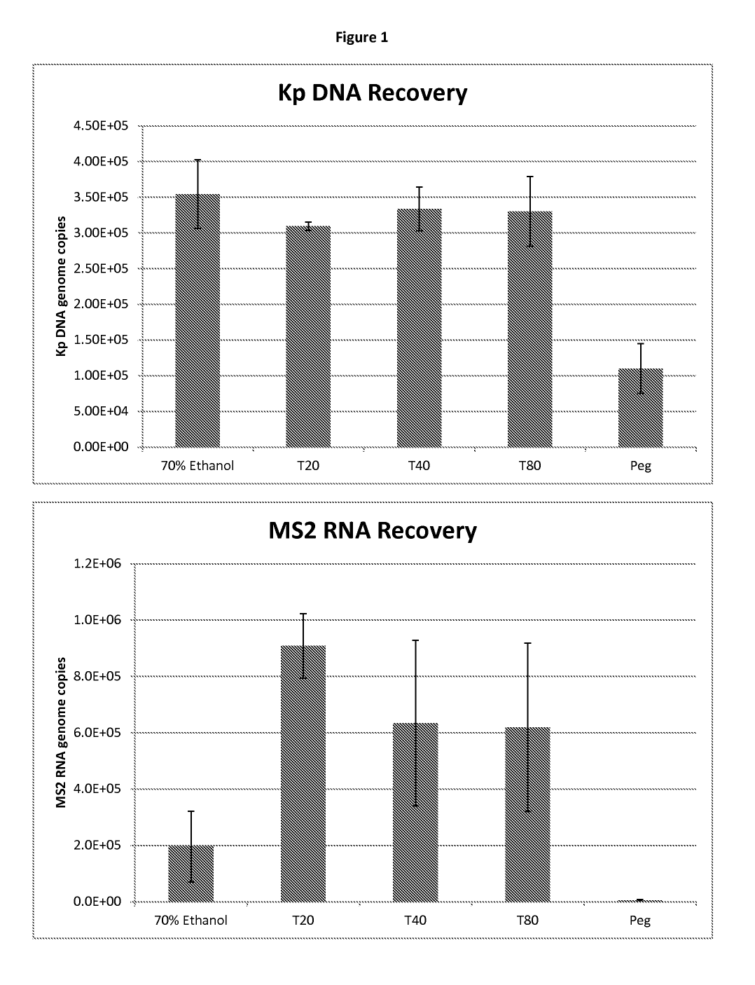

Results are shown in FIG. 1. All three different Tween wash conditions were equivalent or better than 70% ethanol. The PEG8000 wash solution did not perform as well as 70% ethanol and resulted in reduced recovery of nucleic acids.

### Tween 20 Wash Solution Matrix

Various mixtures of Tween 20 and ethanol were tested in a matrix test to determine what ratios of the two would perform equivalent or better than 70% ethanol as a wash solution. Wash solutions with different amounts of Tween 20 and ethanol were made with water. The conditions (A-P) are described in the table below. Additional the standard 70% ethanol wash was run as condition Q.

Ethanol %

| Tween 20% | 35 | 20 | 10 | 0 |
|---|---|---|---|---|
| 40 | A | B | C | D |
| 20 | E | F | G | H |
| 10 | I | J | K | L |
| 5 | M | N | O | P |

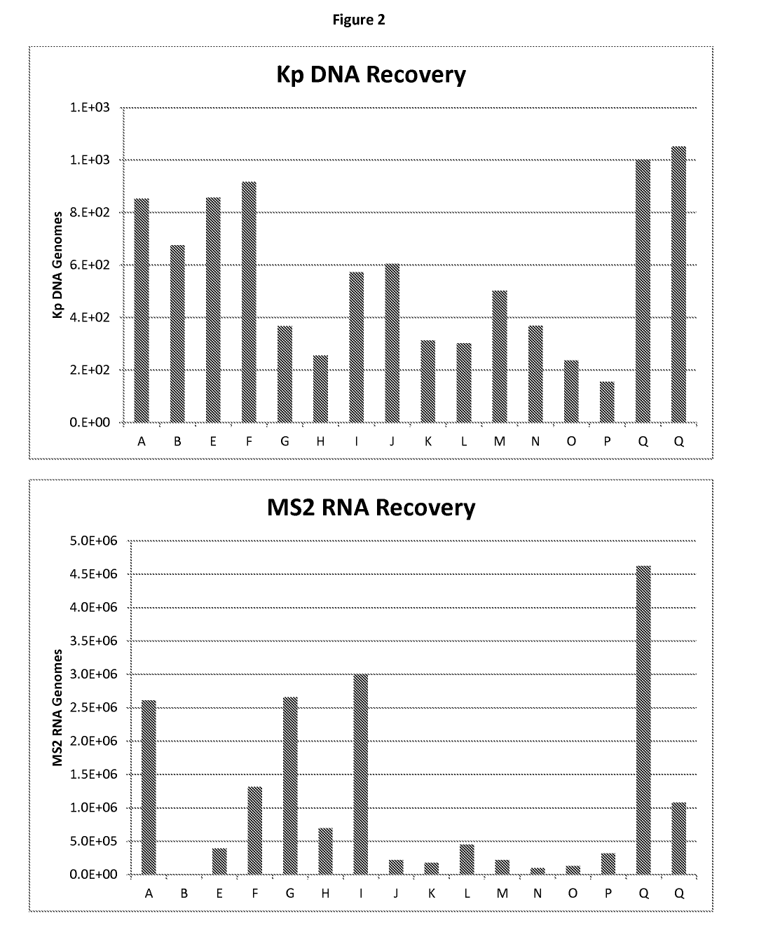

Results are shown in FIG. 2. Conditions C & D were considered too foamy to work as a substitute for the wash solution and were not tested. As in the previous experiment Kp DNA and MS2 RNA were spiked into Primer Dilution Solution and nucleic acids isolated using the SCIP. Samples were eluted with 100 uL of AVE solution.

Conditions A, F, and I were chosen to move forward with as replacements for 70% ethanol wash. Condition A=40% Tween 20; 35% Ethanol, 25% Water; Condition F=20% Tween 20; 20% Ethanol; 60% Water; Condition I=10% Tween 20; 35% Ethanol; 55% Water

### Tween Wash Further Selection

Three different Tween washes were tested further and compared to 70% ethanol wash in the isolation and recovery of nucleic acids. As in previous experiments Kp DNA and MS2 RNA were spiked into Primer Dilution Solution and nucleic acids isolated using the SCIP. Samples were eluted with 100 uL of AVE solution. Sample conditions were tested with n=5 for each.

Wash solutions were denoted by their Tween 20%/Ethanol %. So 40/35 is a wash solution with 40% Tween 20, 35% Ethanol, and 25% water. 0/70 is the normal wash solution of 70% ethanol.

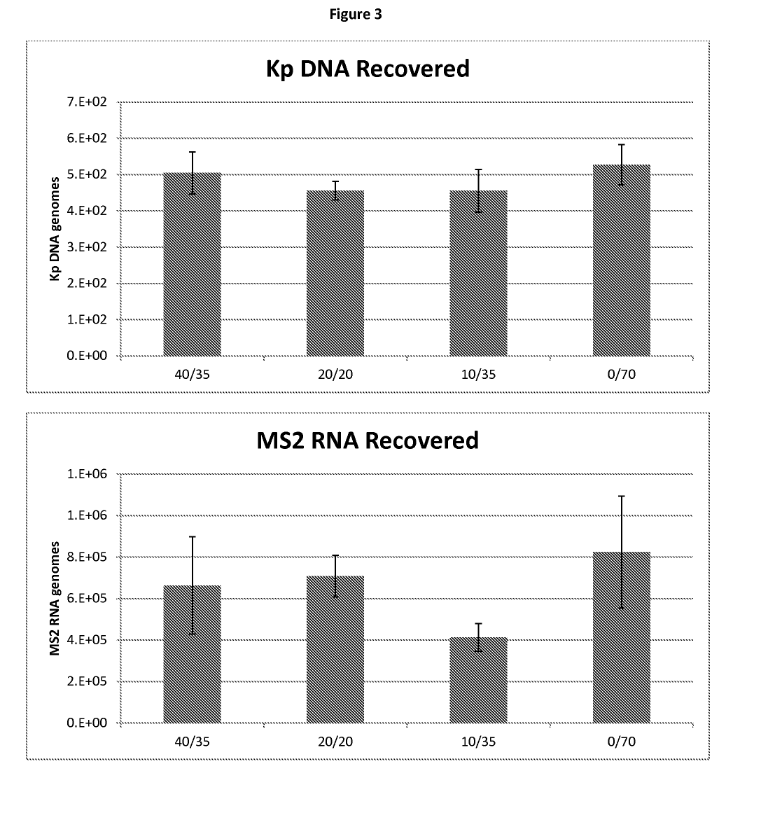

Results are shown in FIG. 3. Both the 40/35 and 20/20 wash performed equivalently to the 70% ethanol wash.

Testing New Wash Solution with Full Lysis and Extraction Protocol

The new wash solutions were tested in a lysis and extraction protocol utilizing whole bacteria and viruses to ensure equivalent performance to the standard 70% ethanol wash. Using Primer Dilution buffer spiked with both Kp bacteria and MS2 virions the following protocol was performed with the exception of the Wash Buffer C1 changed depending on the different formulations being tested (40/35, 20/20, or the standard 0/70).

This is a benchtop protocol for the lysis and extraction of nucleic acids (DNA and RNA) from a variety of cells including bacteria and viruses.
Reagents and consumables
Qiagen DNEasy columns
Qiagen collection tube for columns
Poly A (10 mg/mL) (Ibis MG-00325)
Wash 1 (Abbott MG-00371)
100% Ethanol
Primer Dilution Buffer (Ibis MG-00051)
Covaris S1 (and consumables) or other form of sonication 1.4× A10 Complete
Prepare Lysis Buffer C1: Add 2.5 uL of PolyA per 1 mL of lysis buffer and mix well. Add 100% ethanol to this in a 1:1 ratio.
Prepare Wash Buffer C1: Make up a 70% Ethanol solution mixing 100% ethanol with Primer Dilution buffer in 7:3 ratio.
Prepare 1× A10 Complete: Dilute the 1.4× A10 Complete to 1× with Primer Dilution Buffer.
Protocol:
1. Add 400 uL of Lysis Buffer C1 to 200 uL of sample and mix (A 2:1 ratio of Lysis Buffer C1 to sample).
2. Sonicate the sample (optimized conditions still being worked out; Max Settings at 10 sec will lyse cells in a 30° C. water bath).
3. Load lysate onto DNEasy column. Spin 1 min at 6 k * g.
4. Transfer column to new collection tube and add 500 uL of Lysis Buffer C1 to the column.
5. Spin 1 min at 6 k * g and transfer column to new collection tube.
6. Add 500 uL of Wash Buffer C1 to capture column. Spin 1 min at 6 k * g.
7. Transfer column to new collection tube and add 500 uL of Wash Buffer C1 to the column.
8. Spin 1 min at 6 k * g and transfer column to new collection tube.
9. Spin at full speed for 3 min to dry column.
10. Transfer column to 1.5 mL snapcap tube and add 110 uL of 1× A10 Complete and incubate at room temperature for 3 min.
11. Spin for 1 min at 6 k * g to elute nucleic acids.

Three different Wash Buffer C1 formulations were tested. The standard 70% ethanol (0/70), a 40% Tween 20/35% Ethanol wash solution, and a 20% Tween 20/20% Ethanol solution. Each wash solution was tested in multiple extractions (n=5). The amount of Kp DNA and MS2 RNA recovered was measured by qPCR.

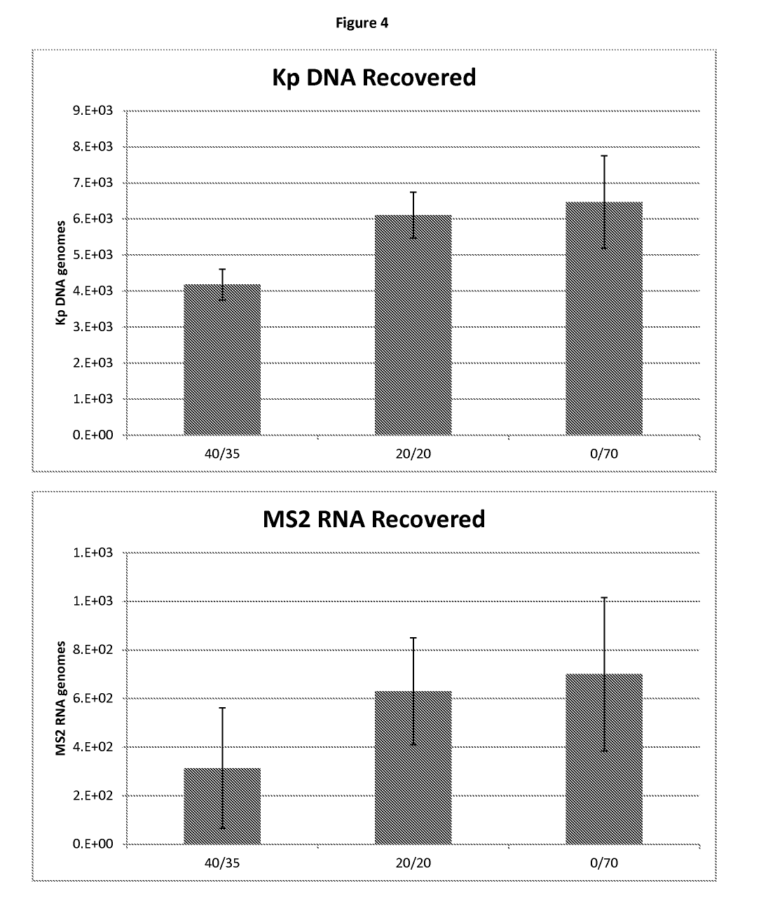

Results are shown in FIG. 4. Again, both new formulations showed equivalent performance to the standard 70% ethanol wash (0/70) demonstrating the new formulations are suitable for a lysis and extraction protocol.

Tween Wash Solution with Adjusted pH

It was observed that the Tween 20 solution that was being used had a low pH. When used to make a wash solution at 20% Tween 20 and 20% ethanol the pH was close to 4. In order to prevent possible damage to the nucleic acids by extreme pH the wash solutions were adjusted to a pH of 5.5 and tested compared to a 70% ethanol wash.

| Sample | Wash buffer |
|---|---|
| 1 | T20P5.5 |
| 2 | T20P5.5 |
| 3 | T20P5.5 |
| 4 | T20P5.5 |
| 5 | T20P5.5 |
| 6 | 0/70 |
| 7 | 0/70 |
| 8 | 0/70 |
| 9 | 0/70 |
| 10 | 0/70 |

### Sample Spike Setup

| 1.00E+07 | cp MS2 per extraction |
|---|---|
| 1.00E+09 | cp MS2 RNA/uL stock |
| | 1:100 dilution MS2 |
| 1.00E+07 | cp MS2 RNA/uL after dilution |
| 1.00E+04 | cp Kp per extraction |
| 5 | fg per Kp genome |
| 5.00E+04 | fg Kp DNA per extraction |
| 5.00E+01 | pg Kp DNA per extraction |
| 10000 | Stock Kp concentration pg/uL |
| | 1:100 dilution of Kp stock |
| 100 | pg/uL Kp after dilution |
| 10 | ug/uL PolyA stock |
| 5 | ug per extraction |

Sample Stock Prep

| | |
|---|---|
| 12 | # of extractions |
| 200 | volume of extraction (uL) |
| 6 | uL PolyA stock |
| 12.00 | uL MS2 RNA dilution |
| 6 | uL Kp DNA dilution |
| 2400 | volume PDB |

The standard column isolation protocol was used to extract the samples.

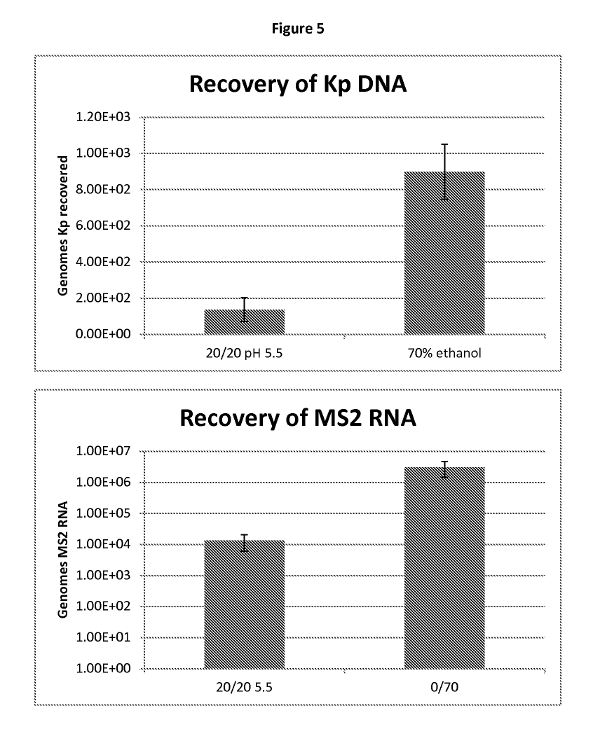

Results are shown in FIG. 5. Recovery of DNA and RNA were both less when the pH of the 20% Tween 20/20% ethanol wash solution was adjusted to a pH of 5.5.

Wash Solution Recovery with Genomic Human DNA

To test the recovery of total DNA when washing with the new tween 20 wash solution compared to ethanol two different 20% Tween 20/20% ethanol solutions were made to test. One was pH 3.5 and a second pH 4.5.

| Stock DNA | 208 | ng/uL |
|---|---|---|
| DNA required per extraction | 5000 | ng |
| Total ng required | 15000 | |
| uL stock required | 72.12 | uL |
| Total spike volume | 600 | uL |
| Spike Prep | | |

| | |
|---|---|
| Mix | 527.88 uL PDB |
| with | 72.12 uL DNA stock |

| Sample | Wash Condition |
|---|---|
| 1 | T20P 3.5 |
| 2 | T20P 4.5 |
| 3 | 0/70 |

1 Add 400 uL Lysis buffer:Ethanol (1:1) to 200 uL spiked sample and mix
2 Transfer lysate to DNEasy column
3 Centrifuge column at 6 k*g for 1 min
4 Transfer the column to a new collection tube
5 Add 500 uL of the appropriate wash buffer (sample table above)
6 Centrifuge column at 6 k*g for 1 min
7 Transfer the column to a new collection tube
8 Add 500 uL of the appropriate wash buffer (sample table above)
9 Centrifuge column at 6 k*g for 1 min
10 Transfer the column to a new collection tube
11 Centrifuge the column at full speed for 3 min to remove any retained wash solution
12 Transfer the column to a clean 1.5 mL snapcap tube and add 100 uL of AVE buffer membrane
13 Incubate at ˜25 C. for 5 min then elute nucleic acid at 6 k*g for 1 min in centrifuge
Following extraction samples were quantitated for total DNA on the Qubit flourometer.
Extracts were also run on a 0.8% agarose gel to compare visually the output. 8 uL of Bionexus All purpose Hi-Lo ladder was also run (lane 1).
Wash solution ng/uL

| | |
|---|---|
| T20P 3.5 | 24 |
| T20P 4.5 | 16 |
| 70% Ethanol | 22 |

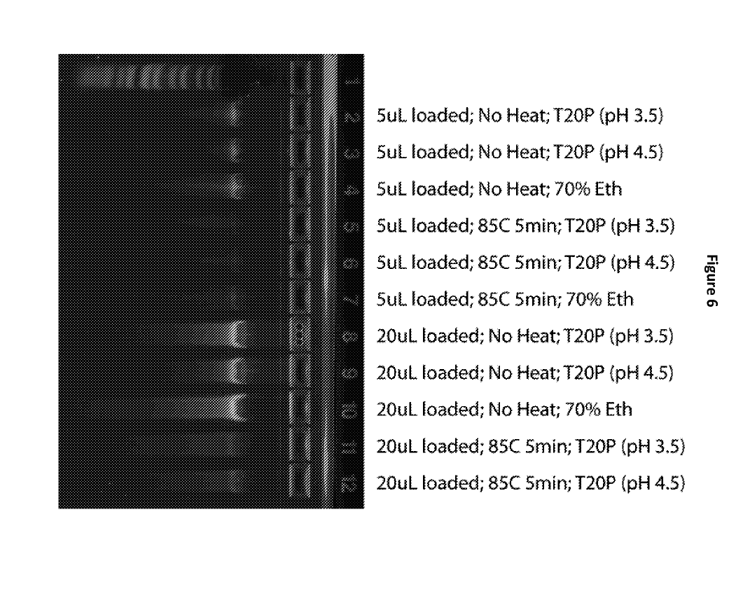

Results are shown in FIG. 6. Recovery of the Tween wash solution at pH 4.5 was less than that of the 70% ethanol. However, the Tween wash solution at pH 3.5 was observed to be equivalent or better than that of 70% ethanol.

Ethanol carryover after nucleic acid column isolation is problematic in downstream applications. To address this issue, new wash buffer formulations of varying Tween 20 and reduced ethanol concentrations (as compared to the 70% Ethanol standard wash solution) were tested for efficacy.

Total nucleic acid (NA) recovery was assessed by performing column isolation of on a mixed sample of Klebsiella pneumonia (Kp) DNA and MS2 bacteriophage virion RNA (DNA/RNA “spike”). Extraction yields were determined by subsequent quantitative real-time polymerase chain reaction (qRT-PCR) for MS2 RNA and qPCR for Kp DNA.

### Column Isolation of Total Nucleic Acids Protocol:

1. The same spike recipe was used for each test condition/wash buffer formulation as follows:

| | |
|---|---|
| 1 uL MS2 RNA stock solution | 1.00E+7 cp MS2 RNA |
| [1.00E+7 copies/uL] | |
| 0.5 uL Kp DNA stock solution | 5.00E+6 pg Kp DNA |
| [1.00E+7 pg/uL] = | total per reaction |
| 0.5 uL Poly dA stock solution | 5.00 ug Poly dA |
| [10 ug/uL] (carrier) | |
| 198 uL Primer Dilution | |
| Buffer (PDB) | |
| 200 uL Total volume per “spike” | |

2. 200 uL spike solution was mixed at a 1:2 ratio with lysis buffer (i.e. 200 uL spike+400 uL lysis buffer), and the full 600 uL sample was applied to a QIAGEN mini spin column and spun at 6,000 rcf for 1 min. to bind NAs
3. Columns were transferred to clean 2.0 mL collection tubes and 500 uL wash buffer was applied. Spin 1′ at 6,000 rcf to wash.
4. Repeat step 3 (wash 2×).
5. Transfer to new collection tube and spin 3′ at maximum speed to remove excess wash buffer.
6. Transfer column to labeled 1.5 mL microfuge tube and apply 110 uL elution buffer AVE (or A9). Allow to stand 3′ to wet column filter, then spin 1′ at 6,000 rcf to elute.
   qPCR and qRT-PCR:

Agilent High ROX II qPCR and QRT-PCR kits were used according to manufacturer specifications and protocols in conjunction with MS2 and Kp primers (IDT) and TaqMan MBG probes (Applied Biosystems) for the qRT-PCR and qPCR assays. Reference RNA for the qRT-PCR was MS2 viral RNA in 10-fold dilutions from 1.00E+8 to 1.00E+1. Reference DNA was Kp DNA in 10-fold dilutions from 10 ng to 1 fg. Assays were run on the Applied Biosystems StepOnePlus real-time PCR system in 25 uL reaction volumes for 45 cycles each using the FAM-MGB reporter.

### Test Wash Buffer Solutions:

Wash buffer formulations tested contained varying concentrations of Tween 20 detergent and ethanol at varying pH levels. Experimental data for each formulation follow: Tx/yP designation: Tx=% v/v Tween 20; y=% v/v ethanol; P=in primer dilution buffer (PDB)

0/70: standard wash buffer (no Tween 20/70% Ethanol in PDB)

### Formulations Tested:

| | | |
|---|---|---|
| T20P (no ethanol) pH 3.5 | T20P pH = 4.5 (no ethanol) | 0/70 |

Nucleic acid (NA) recovery was determined by qRT-PCR for MS2 and qPCR for Kp DNA:

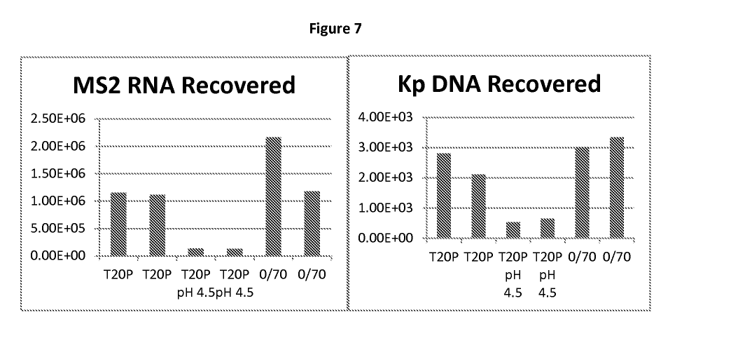

Results are shown in FIG. 7. Using the new wash buffer with no ethanol but at a low pH (3.5) had somewhat similar recovery of DNA and RNA compared to 70% ethanol. However, at a pH of 4.5 it was observed there was reduced recovery.

Additional wash conditions were tested using the following formulations:

| | | | |
|---|---|---|---|
| T20/20 | T20/35 | T30/20 | 0/70 |

NOTE: The “P” designation was dropped, but the base of all solutions henceforth remains PDB.

It was observed that an older bottle of Tween 20 had an altered pH of that of fresh tween 20. In this and all subsequent experiments Tween 20 that was less than 1 year old was used in testing.

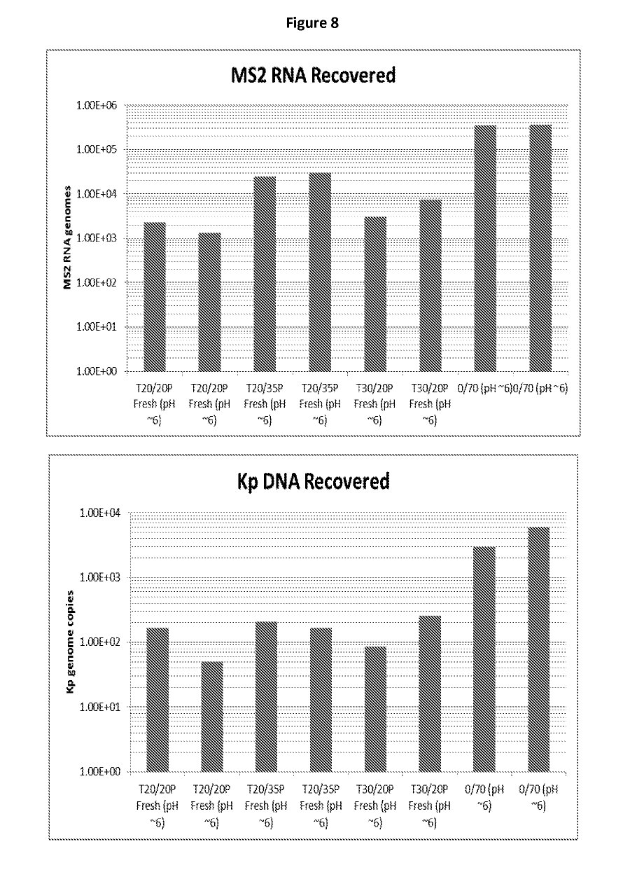

Results are shown in FIG. 8. Nucleic acid recovery was determined by qRT-PCR for MS2 and qPCR for Kp DNA. Again with fresh Tween 20 there was reduced recovery observed at a pH of 6 compared to 70% ethanol.

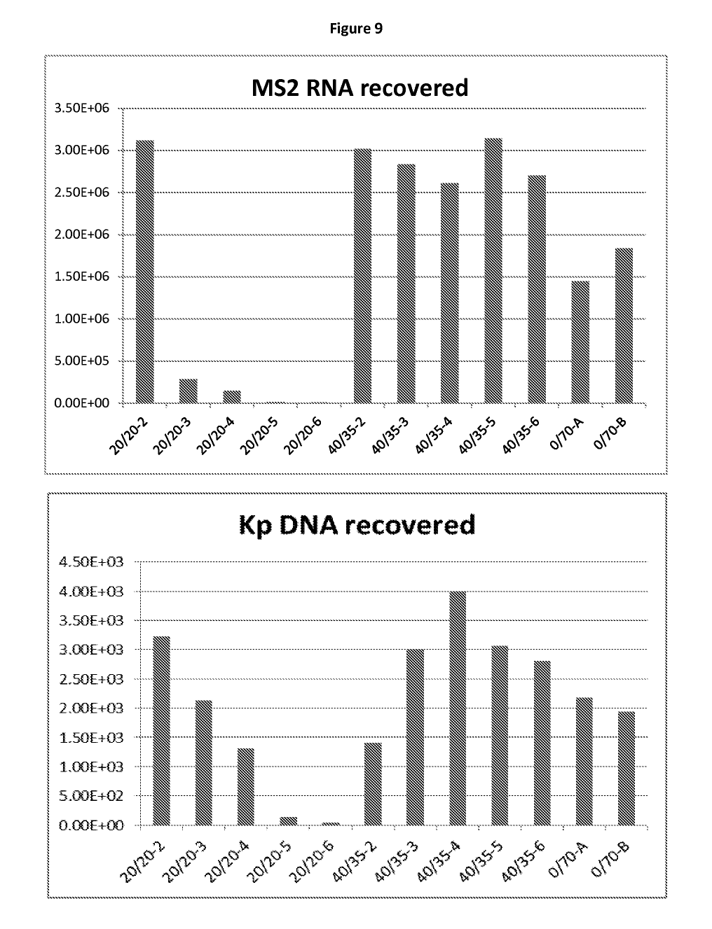

Low pH may affect RNA/DNA quality (e.g. nicking) Wash formulations of T20/20P and T40/35P at pHs ranging from 2-6 were made and qPCR/qRT-PCRs were run to test the effects of pH on total NA recovery yield. Results are shown in FIG. 9. More acidic wash conditions improved yield for both RNA and DNA. Higher Tween and ethanol concentrations improve yield at higher pHs and were more consistently high, in general.

A matrix of wash conditions were tested to find minimal Tween and ethanol concentrations that prove efficient at a moderate pH (5).

### Wash Buffer Formulation Matrix:

| | % Ethanol | | | |
|---|---|---|---|---|
| % Tween 20 | 35 | 30 | 25 | 20 |
| 40 | A | B | C | D |
| 35 | E | F | G | H |
| 30 | I | J | K | L |
| 25 | M | N | O | P |

Q = 0/70 (Standard 70% Ethanol) wash

All solutions pH=5

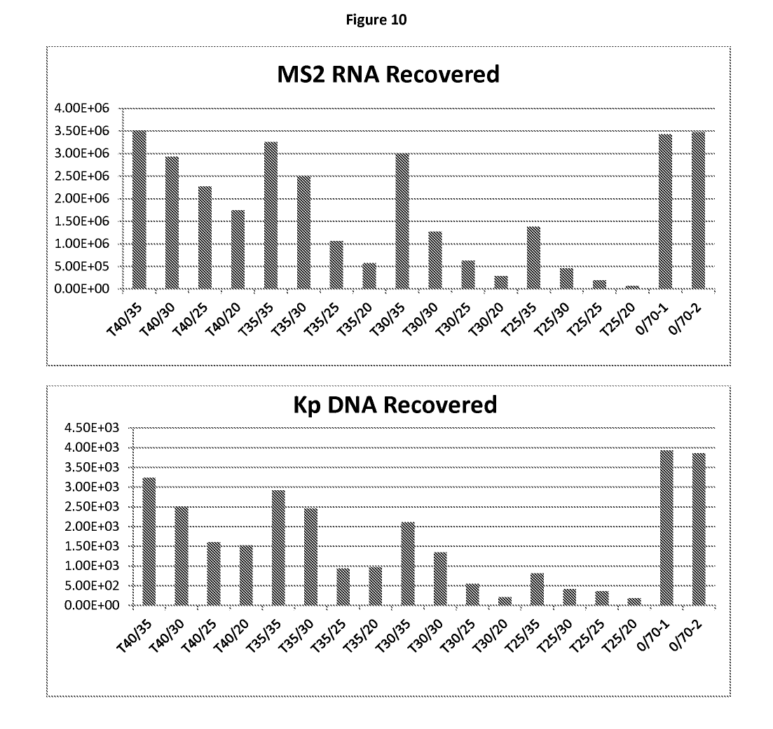

Results are shown in FIG. 10. Consistent with previous results, higher Tween and ethanol concentrations performed better than lower concentrations. Ethanol percent appears to have a more marked effect on efficacy than Tween.

Buffers T40/35 (pH 5) and T35/30 (pH 5) were selected for further testing, and run in quadruplicates against 0/70. Fresh lysates (CC) were prepared in addition to the frozen/purified MS2 RNA/Kp DNA mix (TH) used previously. These samples were also run in quadruplicate, for a total of 8 extractions per wash condition.

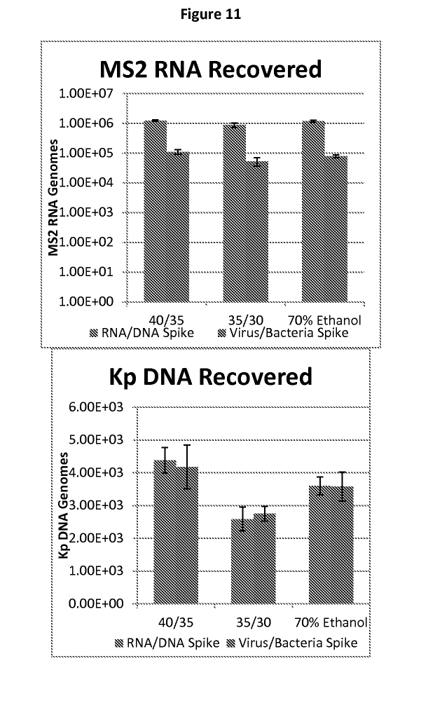

Results are shown in FIG. 11. With the T40/35 wash solution we observed equivalent or better recovery of DNA and RNA compared to 70% ethanol. The T35/30 wash solution had equivanelt performance for RNA but we observed slightly decreased recovery of DNA but was within a 2-fold range difference.

Salts in the wash buffer using varying concentrations of Buffer A9 (1×=12 mM MgCl, 10 mM (NH4)2SO4), as well as 1 mM MgCl and 1 mM KCl in T20/20 wash buffer were tested.

### Wash Formulations Tested:

| | | |
|---|---|---|
| T20/20 + 0.1xA9 | T20/20 + 0.5xA9 | T20/20 + 1.0xA9 |
| T20/20 + 1 mM | T20/20 + 1 mM | |
| KCl (pH = 5) | MgCl (pH = 5) | |

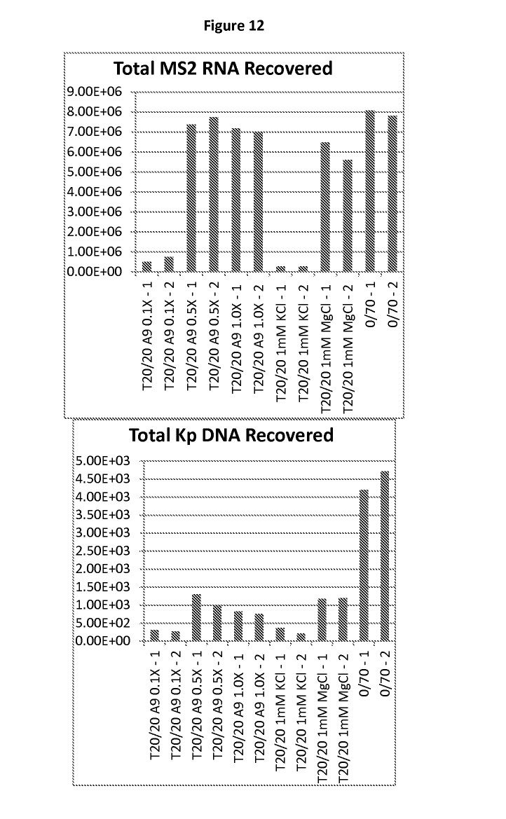

Results are shown in FIG. 12. The addition of KCl had little to no benefit, nor did A9 at 0.1× concentration. However, at 0.5-1.0× A9, RNA yield was roughly equivalent to 0/70. 10 mM MgCl was also effective for the RNA extraction, and performed slightly better in the DNA recovery than either 0.5× or 1.0× A9.

To further test results from the salt tests, new formulations were made using T20/20 and T30/20 with 1 mM and 10 mM MgCl or (NH4)2SO4 added.

### Wash Formulations Tested:

| | | | |
|---|---|---|---|
| T20/20 1 | T20/20 10 | T20/20 1 | T20/20 10 |
| mM MgCl | mM MgCl | mM(NH4)2SO4 | mM (NH4)2SO4 |
| T30/20 1 | T30/20 10 | T30/20 1 | T30/20 10 |
| mM MgCl | mM MgCl | mM(NH4)2SO4 | mM (NH4)2SO4 |

All Wash Wolutions pH=5

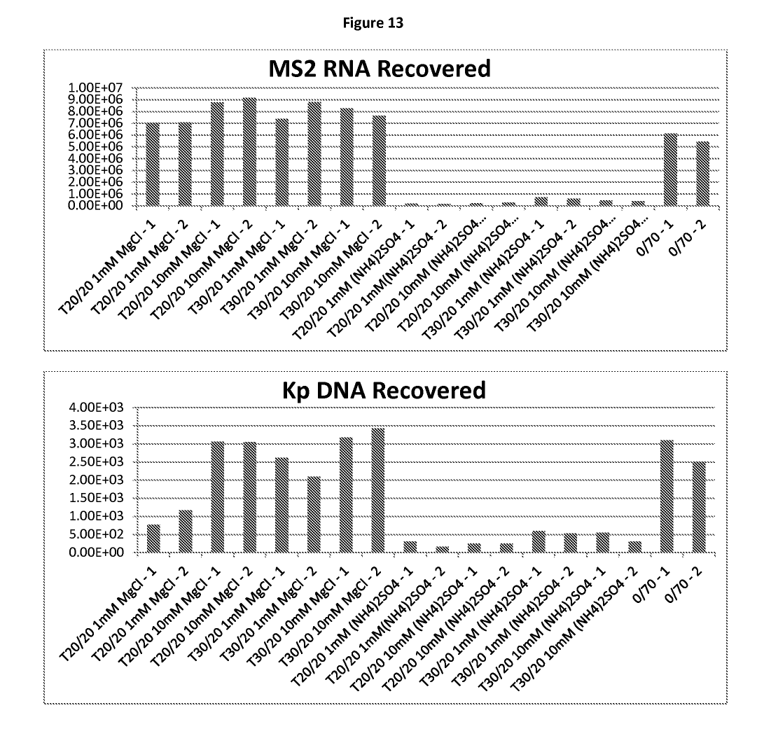

Results are shown in FIG. 13. Ammonium Sulfate ((NH4)2SO4) performed poorly, while the addition of 10 mM MgCl to both T20/20 and T30/20 produced yields slightly higher than the standard 0/70 wash. T20/20+1 mM MgCl buffer was as effective as 0/70 at extracting RNA, though DNA required higher Tween in the T30/20+1 mM MgCl buffer to match 0/70 yields.

In initial tests, the T40/35 wash buffer (with relatively high Tween, high ethanol as compared to other test buffers) performed best. In later tests, the addition of 10 mM MgCl to T20/20 (low Tween, low ethanol) wash buffer produced extraction yields comparable to the standard 0/70 wash. These two “best” formulations were compared using freshly prepared bacterial and viral lysates, as well as frozen stock DNA/RNA isolates.

DNA/RNA spiked samples were performed as described above in Primer Dilution Buffer. The Bacteria/Virus spikes were prepared by spiking K. pneumonia bacteria and MS2 virions into human serum.

The serum samples had a modified protocol for lysis which is described below: 200 uL of the serum spiked with bacteria and virus was mixed with 400 uL of lysis buffer consisting of a 1:1 ratio of Abbott Wash 1 and ethanol. The samples were transferred to a covaris tube and sonicated for 10 sec in a Covaris S. Sonication settings were 10 seconds duration with a duty cycle of 20%, intensity of 10, and cycles per burst 1000. Following sonication the sample was loaded onto the DNEasy column for isolation. The remaining protocol was as per the standard isolation procedure using the appropriate wash solution being tested.

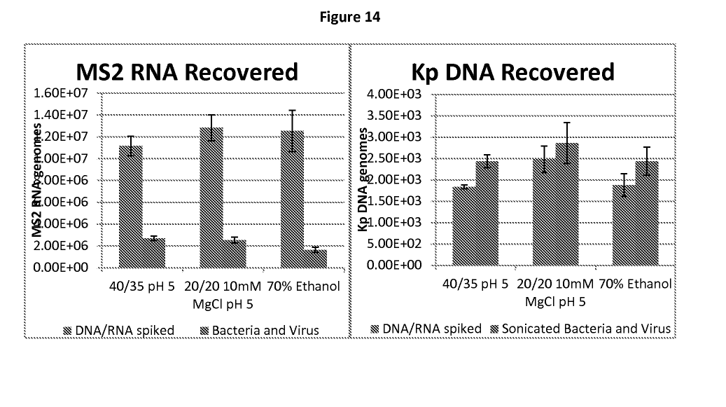

T40/35 (pH=5) vs T20/20+10 mM MgCl. Results are shown in FIG. 14. RNA and DNA yields using T20/20+10 mM MgCl wash buffer were higher than both T40/35 and 0/70. Both the T40/30 pH 5 and the T20/20+10 mM MgCl wash solutions performed equivalent or better than 70% ethanol for recovery of DNA and RNA with both spiked DNA/RNA or from actual bacteria and virus spiked into serum.

An ethanol reduction trial was run to assess how little ethanol would be required to maintain decent recovery with a low Tween 20 percentage (20% or 30%) wash buffer containing 10 mM MgCl. All test wash solutions were pH 5.

### Wash Buffers Tested:

| | | |
|---|---|---|
| T30: T30/20 + 10 mM MgCl | T30/15 + 10 mM MgCl | T30/10 + 10 |
| | | mM MgCl |
| T30/5 + 10 mM MgCl | T30/0 + 10 mM MgCl | |
| T20: T20/20 + 10 mM MgCl | T20/15 + 10 mM MgCl | T20/10 + 10 |
| | | mM MgCl |
| T20/5 + 10 mM MgCl | T20/0 + 10 mM MgCl | |

Extractions were done in duplicate for each wash buffer formulation tested. Data are presented below

individually

averaged for each pair

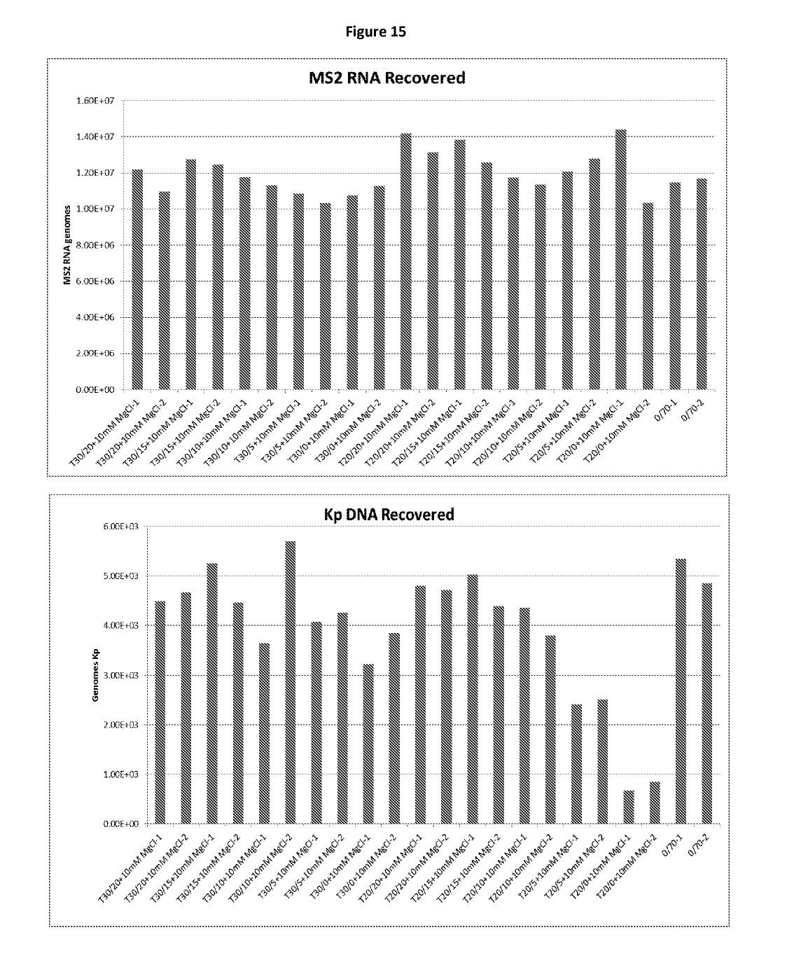

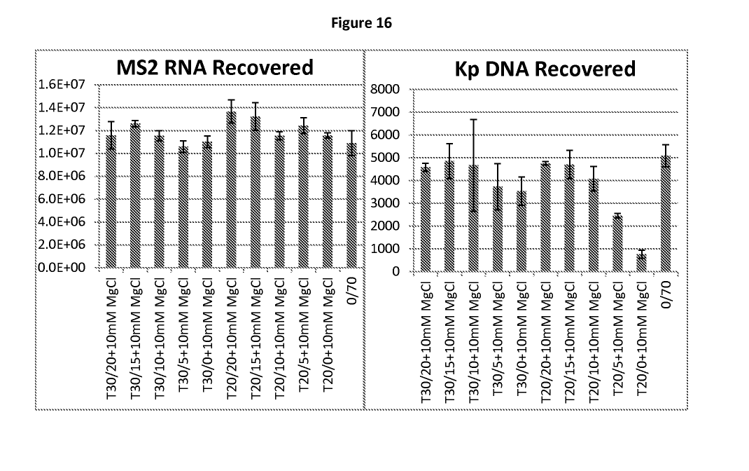

Results are shown in FIGS. 15 and 16. Wash solutions tested down to 10% ethanol showed equivalent recovery of DNA and RNA when compared to the standard 70% ethanol wash solution. Solutions tested with 5% or 0% ethanol had equivalent RNA recovery but showed decreased DNA recovery compared to 70% ethanol.

RNA recovery is excellent (consistently in excess of the standard 0/70 wash) using either 20% or 30% Tween concentrations, as well as in any ethanol concentration tested in the presence of Tween and 10 mM MgCl. DNA yield, however, is more dependent on specific wash buffer formulations.

The three “best” reduced ethanol wash buffer formulations were selected for further testing:

| | | | |
|---|---|---|---|
| T20/10 + 10 | T30/5 + 10 | T30/0 + 10 | pH = 5 |
| mM MgCl | mM MgCl | mM MgCl | for all 3 |

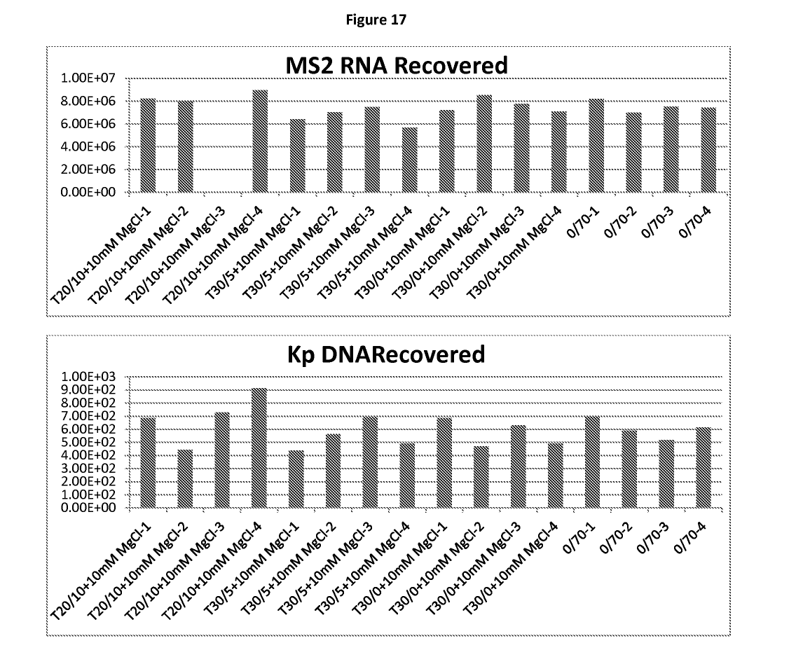

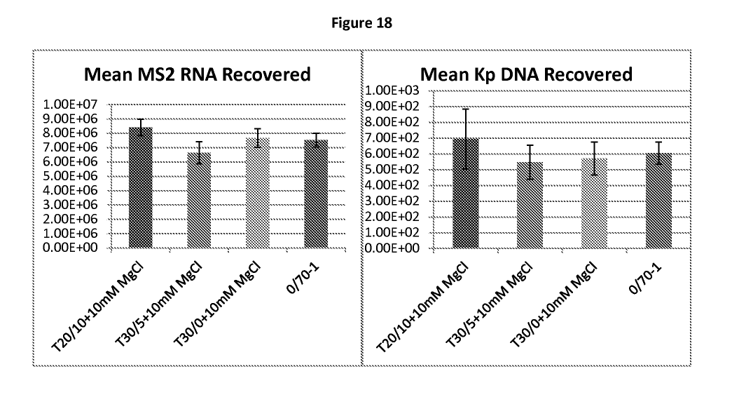

Samples were run in quadruplicate. The qPCR and qRT-PCR results are in FIGS. 17 and 18. All three different wash solutions tested performed equivalent to 70% ethanol.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of isolating nucleic acids, comprising:
a) contacting a sample comprising said nucleic acids with a solid support such that said nucleic acids bind to said solid support;
b) washing said solid support with a wash buffer comprising a surfactant and 35% or less ethanol such that contaminants are removed from said solid support and said nucleic acids are retained on said solid support; and
c) eluting said nucleic acids from said solid support.

2-4. (canceled)

5. The method of claim 1, wherein said wash buffer comprises 10% or less ethanol.

6. The method of claim 1, wherein said wash buffer comprises 5% or less ethanol.

7. (canceled)

8. The method claim 1, wherein said wash buffer comprises between 10% and 50% surfactant.

9. (canceled)

10. The method of claim 1, wherein said surfactant is polysorbate 20.

11. The method of claim 1, wherein wash buffer comprises a salt.

12. The method of claim 11, wherein said salt is MgCl.

13. The method of claim 11, wherein said salt is present at a concentration of between 5 and 15 mM.

14. (canceled)

15. The method of claim 1, wherein said buffer has a pH of between 3.5 and 5.

16. (canceled)

17. The method of claim 1, wherein said buffer comprises components selected from the group consisting of 40% Tween 20, 35% Ethanol, 25% water; 20% Tween 20, 20% Ethanol, 60% water; 10% Tween 20, 35% Ethanol, 55% water; 20% Tween 20, 0% ethanol, 80% water; 35% Tween 20, 30% ethanol, 45% water; 20% Tween 20, 20% ethanol, 60% water+10 mM MgCl; 20% Tween 20, 10% ethanol, 70% water+10 mM MgCl; 30% Tween 20, 5% ethanol, 65% water+10 mM MgCl; and 30% Tween 20, 0% ethanol, 70% water+10 mM MgCl.

18. The method of claim 1, wherein said sample is selected from the group consisting of a cell lysate, blood, serum, and urine.

19. The method of claim 1, wherein said nucleic acid is DNA or RNA.

20. The method of claim 1, wherein said nucleic acid is mammalian, fungal, viral, or bacteria nucleic acid.

21. The method of claim 1, further comprising the step of performing a downstream nucleic acid analysis.

22. A kit, comprising:
a) a wash buffer comprising a surfactant and 35% or less ethanol; and
b) a solid support.

23-26. (canceled)

27. The kit of claim 22, wherein said wash buffer comprises 5% or less ethanol.

28. (canceled)

29. The kit of claim 22, wherein said wash buffer comprises between 10% and 50% surfactant.

30-37. (canceled)

38. The kit of claim 22, wherein said buffer comprises components selected from the group consisting of 40% Tween 20, 35% Ethanol, 25% water; 20% Tween 20, 20% Ethanol, 60% water; 10% Tween 20, 35% Ethanol, 55% water; 20% Tween 20, 0% ethanol, 80% water; 35% Tween 20, 30% ethanol, 45% water; 20% Tween 20, 20% ethanol, 60% water+10 mM MgCl; 20% Tween 20, 10% ethanol, 70% water+10 mM MgCl; 30% Tween 20, 5% ethanol, 65% water+10 mM MgCl; and 30% Tween 20, 0% ethanol, 70% water+10 mM MgCl.

39. The kit of claim 22, wherein said solid support is selected from the group consisting of a resin, a column, a particle, and a bead.

40. A composition, comprising a nucleic acid non-covalently bound to a solid support; and a wash buffer comprising a surfactant and 35% or less ethanol.

41-43. (canceled)
